(19) United States
(12) Patent Application Publication
(10) Pub. No.: US 2011/0262918 A1
(43) Pub. Date: Oct. 27, 2011

(54) IMPROVED NANOPARTICULATE COMPOSITIONS OF POORLY SOLUBLE COMPOUNDS

(75) Inventor: Frank Narz, Witten (DE)

(73) Assignee: Qiagen GmbH

(21) Appl. No.: 13/125,766

(22) PCT Filed: Oct. 22, 2009
(86) PCT No.: PCT/EP09/63890

§ 371 Date: Jul. 18, 2011

(30) Foreign Application Priority Data
Oct. 23, 2008 (EP) 08167412.9

(51) Int. Cl. C12Q 1/68; C07H 21/02

(52) U.S. Cl. 435/6.11; 536/23.1

(57) ABSTRACT

The present invention is related to a method for the quantification of one or more target ribonucleic acids in a sample comprising the steps of, (i) providing a sample comprising said one or more target ribonucleic acids, (ii) contacting said sample with a ribonucleic acid specific fluorescence dye under conditions allowing for binding of said dye to the one or more ribonucleic acids in said sample, (iii) measuring fluorescence of said RNA-bound dye in said sample, (iv) correlating said measured fluorescence to the total amount of RNA in the sample, (v) reverse transcribing said one or more ribonucleic acids, thereby creating double-stranded nucleic acids, (vi) amplifying said one or more created double-stranded nucleic acids, wherein one or more fluorescence probes specific for said one or more amplification products are present during and/or after amplification under conditions allowing for binding of said one or more probes to said one or more created double-stranded deoxyribonucleic acids in the sample, (vii) measuring fluorescence of said one or more probes bound to said one or more amplification products during and/or after the amplification reaction and correlating said measured fluorescence to the amount of target RNA sequence in the sample and (viii) normalizing the amount of target RNA sequence in the sample to the total amount of RNA in the sample.

### TECHNICAL FIELD OF THE INVENTION

The present invention is in the field of Biology and Chemistry. In particular, the invention is in the field of Molecular Biology. More particular, the invention is in the field of quantification of nucleic acids and real-time PCR. Furthermore, the invention is related to normalized quantification of target ribonucleic acids.

### BACKGROUND OF THE INVENTION

The quantification (quantitation) of specific target ribonucleic acids (also specific RNAs herein) in mixtures of nucleic acids is of importance in a number of applications in is molecular biology, such as gene expression analysis or during purification of specific RNAs from a mixture of nucleic acids. In quantification methods, the concentrations and/or the relative or absolute amounts of specific RNAs in samples are determined. In particular, for the analysis of gene expression, for example for measuring mRNA levels in biological samples, a reproducible and comparative method is desired. For example, it is not always possible to obtain biological samples with comparable volume, amount of nucleic acid, cellular material or the like.

In addition, sensitivity and selectivity of detection and quantification of nucleic acids in biological samples is of importance. For better comparison of the quantities of specific RNAs in two or more different (biological) samples or the comparison of the quantities of two or more different specific RNAs in a sample, a normalization of the quantities of the specific RNAs to the input nucleic acids or a specific class of input nucleic acid has to be performed. Quantities of specific RNAs can, e.g., be normalized by relating these quantities to an internal standard of the sample or to the overall, i.e. total, amount of nucleic acid or to the amount of a specific class of nucleic acid in the sample.

For conventional quantification of nucleic acids in (biological) samples, quantitative (real-time) PCR (qPCR) is widely used. For RNA, particularly mRNA, quantitative real-time reverse transcription PCR(RT-qPCR) is used in this field. Different approaches for the normalization of data obtained from quantitative PCR methods have been employed. Among them is the normalization of the quantities of specific mRNAs to the quantities of one or more mRNAs of different reference genes, e.g. housekeeping or maintenance genes, such as to beta-actin, glyceraldehyde-3-phosphate dehydrogenase (GAPDH), hypoxanthine-guanine phosphoribosyl transferase (HPRT), or 28S or 18S ribosomal RNA. However, the expression levels of such normalizer genes have been shown to vary depending on experimental conditions, preparation and source (e.g. tissue or cell type) of the samples and therefore they are not reliably indicative for the input nucleic acids. It is therefore commonly required to test a range of different housekeeping genes in a laborious and error-prone procedure in order to identify those which do not change between samples under investigation.

Other approaches, for example, rely on the normalization to the total content of DNA and/or RNA or the total content of e.g. ribosomal RNA (rRNA). As the content of ribosomal RNA in biological cells and samples is also subject to variations depending on a variety of factors, normalization to rRNA is also less preferred. State of the art methods relying on the normalization to e.g. total nucleic acid content, total RNA content or total content of genomic DNA are also limited, e.g. by variations in these contents or the quality of the nucleic acid samples. Normalization to alien or artificial molecules, e.g. in vitro transcripts, that have been incorporated into a sample (e.g. a cell extract or a sample derived from a tissue) is also not in all cases an adequate procedure, since they do not represent the nucleic acid (e.g. genomic DNA, RNA, mRNA) content in a cell.

Besides, for comparison of normalized data and reproducibility of the experimental procedures, thorough documentation of the applied experimental conditions is required. This is particularly relevant, when the quantities of the nucleic acid of interest and the normalizer nucleic acid are determined separately or using different methods.

Therefore, the technical problem underlying the present invention was to develop and to provide an improved, in particular a less laborious and error-prone, method for the normalization of quantities of target ribonucleic acids.

### SUMMARY OF THE INVENTION

The invention relates to a method for the quantification of one or more target ribonucleic acids in a sample comprising the steps of:
(i) providing a sample comprising said one or more target ribonucleic acids;
(ii) contacting said sample with a ribonucleic acid specific fluorescence dye under conditions allowing for binding of said dye to the one or more ribonucleic acids in said sample;
(iii) measuring fluorescence of said RNA-bound dye in said sample;
(iv) correlating said measured fluorescence to the total amount of RNA in the sample;
(v) reverse transcribing said one or more ribonucleic acids, thereby creating double-stranded nucleic acids;
(vi) amplifying said one or more created double-stranded nucleic acids, wherein one or more fluorescence probes specific for said one or more amplification products are present during and/or after amplification under conditions allowing for binding of said one or more probes to said one or more created double-stranded nucleic acids in the sample;
(vii) measuring fluorescence of said one or more probes bound to said one or more amplification products during and/or after the amplification reaction and correlating said measured fluorescence to the amount of target RNA sequence in the sample;
(viii) normalizing the amount of target RNA sequence in the sample to the total amount of RNA in the sample.

A sample contains at least nucleic acid molecules comprising the ribonucleic acid to be quantified. The nucleic acids can be embedded in cells or organisms but can also be present in a cell free system. A sample may be a fluid, a lysate, solid matrix or anything else that contains nucleic acid molecules. A sample in the meaning of the invention can be all biological tissues and all fluids such as lymph, urine, cerebral fluid. Tissues may be, e.g. epithelium tissue, connective tissue such as bone or blood, muscle tissue such as visceral or smooth muscle and skeletal muscle and, nervous tissue. In one embodiment the sample is a cell culture or an extract from a cell culture.

Herein, a target ribonucleic acid may be of any origin, e.g. viral, bacterial, archae-bacterial, fungal, ribosomal, eukaryotic or prokaryotic. It may be RNA from any biological sample and any organism, tissue, cell or sub-cellular compartment. It may e.g. be nucleic acid from a plant, a fungus, an animal, and particularly human nucleic acid. The RNA may be pre-treated before quantification, e.g. by isolation, purification or modification. Also artificial RNAs may be quantified. The length of the RNAs may vary. The RNAs may be modified, e.g. may comprise one or more modified nucleobases or modified sugar moieties (e.g. comprising methoxy groups). The backbone of the RNA may comprise one or more peptide bonds as in peptide nucleic acid (PNA). The RNA may comprise base analoga such as non-purine or non-pyrimidine analoga or nucleotide analoga. It may also comprise additional attachments such as proteins, peptides and/or or amino acids.

A “primer” herein refers to an oligonucleotide comprising a sequence that is substantially complementary to a nucleic acid to be transcribed (“template”). During replication polymerases attach nucleotides to the 3′ end of the primer substantially complementary to the respective nucleotides of the template.

An “RNA specific dye” herein is defined as follows:

Spectral properties of the “RNA specific dye” must not interfere with the spectral properties used for detection of the target RNA, allowing simultaneous detection. The reaction mix used for the detection of the target RNA contains various substance that can interfere with the measurement of RNA, e.g. DNA, nucleotides, oligonucleotides, proteins, detergents, salts. The RNA specific dye of choice must therefore not be influenced by such substances to a grade that disturbs the quantification of RNA. There needs to be a correlation between RNA concentration and fluorescence in the presence of the above substances.

An “RNA specific dye” may be a dye that specifically binds to RNA and substantially not to other nucleic acids such as DNA or other components typically present in the sample. Upon binding, the RNA specific dye may change its spectral properties, e.g. an increase in fluorescence. Alternatively, the “RNA specific dye” may also be a dye that unspecifically binds nucleic acids in the sample but significant changes of spectral properties such as fluorescence are only observed upon binding to RNA. Thus, an “RNA specific dye” allows for the selective detection of RNA without being significantly interfered by other nucleic acids or proteins, detergents, salts or other components typically present in the sample.

A “DNA specific dye” herein is defined as follows:

Spectral properties of the “DNA specific dye” must not interfere with the spectral properties of the dye used for detection of total RNA, allowing simultaneous detection. The “DNA specific dye” needs to be specific for the amplification product, either by choosing a dye selective for double stranded DNA or by a choosing a probe selective for the sequence of the amplified products.

A “fluorescence probe” herein is either a DNA specific dye or a nucleic acid probe labeled with a fluorescent dye. A nucleic acid probe according to the present invention is an oligonucleotide, nucleic acid or a fragment thereof, which is substantially complementary to a specific nucleic acid sequence.

The RNA specific dye differs in spectral property from the DNA specific dye or the fluorescent dye such that they may both be detected.

The invention also relates to a kit for the quantification of target RNA in a sample comprising, (i) a fluorescence dye which specifically binds to RNA and (ii) one or more fluorescence probes specific for one or more DNA amplification products.

As used herein, a kit is a packaged combination optionally including instructions for use of the combination and/or other reactions and components for such use.

The invention also relates to the use of an RNA specific fluorescence dye such as Quant-iT™-RNA in the normalization of the amount of a target RNA sequence in a sample to the total amount of RNA in the sample.

### DETAILED DESCRIPTION OF THE INVENTION

The Inventors have found that certain dyes allow for RNA normalization. The invention relates to a method for the quantification of one or more target ribonucleic acids in a sample comprising the steps of:
(i) providing a sample comprising said one or more target ribonucleic acids;
(ii) contacting said sample with a ribonucleic acid specific fluorescence dye under conditions allowing for binding of said dye to the one or more ribonucleic acids in said sample;
(iii) measuring fluorescence of said RNA-bound dye in said sample
(iv) correlating said measured fluorescence to the total amount of RNA in the sample;
(v) reverse transcribing said one or more ribonucleic acids, thereby creating double-stranded nucleic acids;
(vi) amplifying said one or more created double-stranded nucleic acids, wherein one or more fluorescence probes specific for said one or more amplification products are present during and/or after amplification under conditions allowing for binding of said one or more probes to said one or more created double-stranded nucleic acids in the sample;
(vii) measuring fluorescence of said one or more probes bound to said one or more amplification products during and/or after the amplification reaction and correlating said measured fluorescence to the amount of target RNA sequence in the sample;
(i) normalizing the amount of target RNA sequence in the sample to the total amount of RNA in the sample.

In one embodiment of the method the sample is total RNA preparation. In another particular embodiment, the target RNA to be quantified is RNA selected from the group consisting of mRNA, rRNA, tRNA, nRNA, siRNA, snRNA, snoRNA, scaRNA, microRNA, dsRNA, ribozyme, riboswitch and viral RNA and the total quantity of RNA is selected from the group consisting of the total quantity of RNA, the total quantity of mRNA, the total quantity of rRNA, the total quantity of tRNA, the total quantity of nRNA, the total quantity of siRNA, the total quantity of snRNA, the total quantity of snoRNA, the total quantity of scaRNA, the total quantity of microRNA, the total quantity of dsRNA, the total quantity of ribozyme, the total quantity of riboswitch, the total quantity of viral RNA.

Preferably, the target RNA to be quantified is mRNA.

In a preferred embodiment, the RNA specific fluorescence dye is selected from the group consisting of:

### Compound 6:

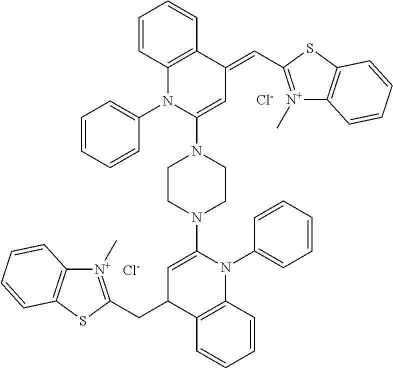

### Compound 11:

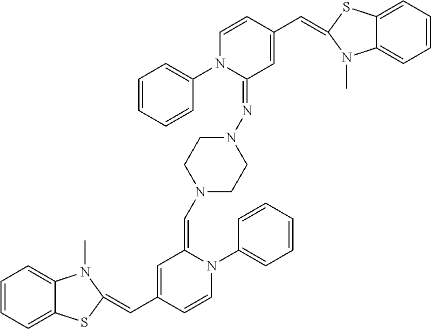

### Compound 19:

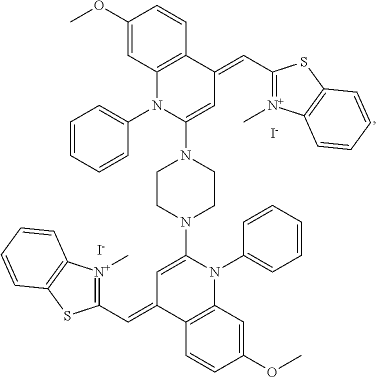

### Compound 20:

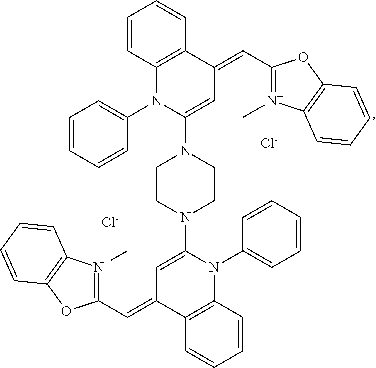

### Compound 23:

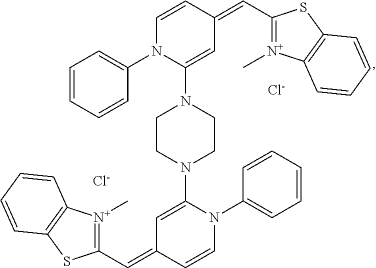

The compounds as described above have been published in US 2008/0199875 A1 on pages 14 to 16. (See compounds 6, 11, 19, 20 and 23).

In a specifically preferred embodiment, the RNA specific fluorescence dye in the present invention is compound 11.

In a further specifically preferred embodiment, the RNA specific fluorescence dye in the present invention is Quant-iT™-RNA reagent (see US 2008/0199875 A1).

The fluorescence probe specific for DNA preferably is a fluorescence dye specific for double-stranded DNA. Such a dye herein may be selected from the group of, SYTO-9, SYTO-13, SYTO-16, SYTO-64, SYTO-82, YO-PRO-1, SYTO-60, SYTO-62, SYTOX Orange, SYBR Green I, TO-PRO-3, TOTO-3, POPO-3, ethidiumbromide and BOBO-3. The Toto family dyes are not as suited as the other dyes.

Ideally, the fluorescence dye for DNA is SYBR Green I (2-{2-[(3-Dimethylaminopropyl)-propylamino]-1-phenyl-1H-chinolin-4-ylidenmethyl}-3-methyl-benzothiazol-3-ium cation).

In a further embodiment the fluorescence probe specific for DNA is an oligonucleotide probe labelled with a fluorescence dye, wherein the oligonucleotide probe is substantially complementary to a sequence on the DNA created from the target ribonucleic acid molecule. Particularly, the fluorescently labelled probes are labelled with a dye selected from the group consisting of FAM, VIC, NED, Fluorescein, FITC, IRD-700/800, CY3, CY5, CY3.5, CY5.5, HEX, TET, TAMRA, JOE, ROX, BODIPY TMR, Oregon Green, Rhodamine Green, Rhodamine Red, Texas Red, Yakima Yellow, Alexa Fluor and PET.

In one embodiment two or more target ribonucleic acids are quantified in the sample and an oligonucleotide probe labelled with a different fluorescent dye is used for each target RNA to be quantified. Ideally this probe is complementary to its target sequence. However, also mismatches may be desired in some cases. Such a probe may also have a tail or end sequence which does not bind the target sequence.

A skilled person knows how to choose the length and sequence of the probes and primers depending on the reaction temperature of the elongation reaction, on the particular enzyme(s) used (e.g. thermostable polymerase, reverse transcriptase) and on the sequence of the respective binding partners. In particular, the hybridization probe is a LightCycler probe (Roche) or the hydrolysis probe is a TaqMan probe (Roche). In other embodiments the hairpin probe is selected from the group consisting of molecular beacon, Scorpion primer, Sunrise primer, LUX primer and Amplifluor primer.

In some embodiments of the invention quantification comprises a nucleic acid amplification reaction such as the non-isothermal amplification methods polymerase chain reaction (PCR), particularly quantitative real-time PCR or isothermal amplification methods such as NASBA (nucleic acids sequence based amplification), TMA (Transcription mediated amplification), 3SR (self-sustained sequence amplification), SDA (Strand displacement amplification), HDA (helicase dependent amplification, with heat-labile or heat-stabile enzymes), RPA (recombinase polymerase amplification), LAMP (Loop-mediated amplification); or SMAP (SMart Amplification Process) These technologies make use of a couple of different enzymes, proteins, primers and accessory molecules that are well known for persons skilled in the art. The polymerases include polymerases selected from the group comprising Thermus thermophilus (Tth) DNA polymerase, Thermus acquaticus (Taq) DNA polymerase, Thermotoga maritima (Tma) DNA polymerase, Thermococcus litoralis (Tli) DNA polymerase, Pyrococcus furiosus (Pfu) DNA polymerase, Pyrococcus woesei (Pwo) DNA polymerase, Pyrococcus kodakaraensis KOD DNA polymerase, Thermus filiformis (Tfi) DNA polymerase, Sulfolobus solfataricus Dpo4 DNA polymerase, Thermus pacificus (Tpac) DNA polymerase, Thermus eggertsonii (Teg) DNA polymerase and Thermus flavus (Tfl) DNA polymerase and the polymerases of phages e.g. Phi29-phage, Phi29 like phages such as Cp-1, PRD-1, Phi 15, Phi 21, PZE, PZA, Nf, M2Y, B103, SF5, GA-1, Cp-5, Cp-7, PR4, PR5, PR722, or L 17. The polymerases include also polymerase form other organism such as from E. coli, T4, T7. Other additional proteins may improve the methods, for example helicases, single-stranded binding proteins, or other DNA-binding proteins, and recombinases.

It is preferred that reverse transcribing the target RNA and amplifying the created DNA is performed in a polymerase chain reaction.

In some embodiments of the invention reverse transcribing and quantifying are performed in the same reaction container.

The reverse transcriptase may be a polymerase also used for amplification during the quantification steps.

The enzyme having reverse transcriptase activity in the context of the invention may be of different origin, including viral, bacteria, Archae-bacteria and eukaryotic origin, especially originating from thermostable organisms. This includes enzymes originating from introns, retrotransposons or retroviruses. An enzyme with reverse transcriptase activity in the context of the invention is an enzyme which is able to add to the 3′ end of a desoxyribonucleic acid or a ribonucleic acid, hybridized to a complementary desoxyribonucleic acid or a ribonucleic acid or vice versa, one or more desoxyribonucleotides at suitable reaction conditions, e.g. buffer conditions, complementary to said desoxyribonucleic acid or a ribonucleic acid. This includes enzyme, that intrinsically have reverse transcriptase, but also enzymes that were evolved or mutated in their gene sequence to gain such function or that gain such function by adjusting buffer or other reaction parameters.

Preferably, the enzyme having reverse transcriptase activity in the context of the invention is selected from the group comprising HIV reverse transcriptase, M-MLV reverse transcriptase, EAIV reverse transcriptase, AMV reverse transcriptase, Thermus thermophilus DNA Polymerase I, M-MLV RNase H, Superscript, Superscript II, Superscript III, Monstersript (Epicentre), Omniscript, Sensiscript Reverse Transcriptase (Qiagen), ThermoScript and Thermo-X (both Invitrogen). The enzyme may also have increased fidelity like e.g. AccuScript reverse Transcriptase (Stratagene). A skilled person knows that one or more suitable enzyme with reverse transcriptase activity can be mixed to gain optimized conditions or novel features. This may include amongst others mixtures of e.g. a mesophilic and a thermophilic enzymes, or an enzyme having RNase H activity and an enzyme being RNase H negative, or an enzyme with increased fidelity and an thermophilic enzyme. Numerous other combinations are possible based on the list of preferred enzymes having reverse transcriptase activity in the scope of the invention.

As indicated herein above, for the analysis of gene expression, the quantification of mRNA in samples can be performed using quantitative real-time reverse transcription PCR (RT-qPCR). RT-qPCR methods employ a combination of three steps: (i) the reverse transcription of the mRNA into cDNA using a RNA-dependent DNA polymerase (i.e. a reverse transcriptase), (ii) the amplification of cDNA using PCR, and (iii) the detection and quantification of the amplification products in real time. For reverse transcription and PCR-based amplification, dNTPs (“nucleotide mixture”) need to be present in the reaction buffer. A nucleotide mixture according to the present invention is a mixture of dNTPs, i.e. a mixture of dATP, dCTP, dGTP and dTTP/dUTP suitable for the use in PCR. For particular embodiments of the present invention the relative amounts of these dNTPs may be adapted according to the particular nucleotide content of the template nucleic acids. The RT-qPCR steps can either be performed in a single-stage process or in a two-stage process. In the first case, reverse transcription and PCR-based amplification are performed in the same reaction container, e.g. by utilizing a DNA polymerase which has intrinsic reverse transcription functionality, like Thermus thermophilus (Tth) polymerase. In a two-stage setup the steps of reverse transcribing the RNA and amplifying the DNA are performed separately, e.g. in different reaction containers. The steps of the methods according to the present invention may be conducted in suitable reaction buffers, e.g. comprising salts such as magnesium ions. As already stated, the different steps may or may not be conducted in the same buffers and reaction containers. In contrast to RT-qPCR, in qPCR no reverse transcription is performed, therefore it is a quantification method for DNA rather than for RNA.

The reverse transcription of (m)RNA in RT-qPCR and the amplification of (c)DNA in qPCR and RT-qPCR need to be primed by oligonucleotides (“primers”). In the case of mRNA quantification with RT-qPCR, mRNA specific oligonucleotides can be used, e.g. oligo-dT primers that hybridize to the poly-A-tail of mRNA. However, also random primers of varying lengths can be utilized.

The quantifying steps may in some embodiments comprise a method selected from the group consisting of gel electrophoresis, capillary electrophoresis, labelling reactions with subsequent detection measures and quantitative real-time PCR. Preferably, quantification comprises quantitative real-time PCR or quantitative real-time reverse transcription PCR. In preferred embodiments of the invention, the quantification step(s) comprise(s) (i) the reverse transcription of RNA (e.g. mRNA) into DNA (e.g. cDNA) using a RNA-dependent DNA polymerase (i.e. a reverse transcriptase), (ii) the amplification of the DNA produced by reverse transcription using PCR, and (iii) the detection and quantification of the amplification products in real time. It is particularly preferred if the polymerase chain reaction is a quantitive real-time PCR. Selective primers are used in quantitative real-time PCR to quantify the target RNA.

In some embodiments of the invention, the quantification of the target RNA sequence involves the use of an oligonucleotide probe labelled with one or more fluorescent dye(s) and/or quenchers during quantitative real-time PCR. The fluorescently labelled nucleic acid probes may for example be selected from the group consisting of hybridization probe, hydrolysis probe and hairpin probe.

Moreover, standard quantitative real-time PCR protocols and kits can be adapted or amended for the means and methods according to the present invention.

As mentioned above, real-time PCR (also designated herein as quantitative PCR or quantitative real-time PCR (qPCR)) is a method to simultaneously amplify and quantify nucleic acids using a polymerase chain reaction (PCR). Quantitative real-time reverse transcription PCR (RT-qPCR) is a quantitative real-time PCR method further comprising a reverse transcription of RNA into DNA, e.g. mRNA into cDNA. In qPCR and RT-qPCR methods, the amplified nucleic acid is quantified as it accumulates. Typically, fluorescent dyes that intercalate with double-stranded DNA (e.g. ethidiumbromide or SYBR® Green I) or modified nucleic acid probes (“reporter probes”) that fluoresce when hybridized with a complementary nucleic acid (e.g. the accumulating DNA) are used for quantification in qPCR based methods. Particularly, fluorogenic primers, hybridization probes (e.g. LightCycler probes (Roche)), hydrolysis probes (e.g. TaqMan probes (Roche)), or hairpin probes, such as molecular beacons, Scorpion primers (DxS), Sunrise primers (Oncor), LUX primers (Invitrogen), Amplifluor primers (Intergen) or the like can be used as reporter probes. In accordance with the present invention, fluorogenic primers or probes may for example be primers or probes to which fluorescence dyes have been attached, e.g. covalently attached. Such fluorescence dyes may for example be FAM (5- or 6-carboxyfluorescein), VIC, NED, Fluorescein, FITC, IRD-700/800, CY3, CY5, CY3.5, CY5.5, HEX, TET, TAMRA, JOE, ROX, BODIPY TMR, Oregon Green, Rhodamine Green, Rhodamine Red, Texas Red, Yakima Yellow, Alexa Fluor, PET and the like. Particular reporter probes may additionally comprise fluorescence quenchers.

The invention also relates to a kit for the quantification of target RNA in a sample comprising: (i) a fluorescence dye which specifically binds to RNA and (ii) one or more fluorescence probes specific for one or more DNA amplification products. In preferred embodiments of the invention, the kit additionally comprises a polymerase. The kit may additionally also comprise a nucleotide mixture and (a) reaction buffer(s). In some embodiments the kit additionally comprises a reverse transcriptase.

Preferred RNA specific dyes of the present invention are disclosed in particular in US2008/0199875 A1. As outlined above, particularly preferred RNA specific dyes in the context of the present invention are compounds 6, 11, 19, 20 and 23 as described on pages 14 to 16 of US 2008/0199875 A1.

In a specifically preferred embodiment, the RNA specific fluorescence dye in the present invention is compound II.

In a further specifically preferred embodiment, the RNA specific fluorescence daye in the present invention is Quant-iT™-RNA reagent (see US 2008/0199875 A1).

The invention relates to the use of an RNA specific fluorescence dye, such as but not limited to Quant-iT™-RNA reagent, in the normalization of the amount of a target RNA in a sample to the total amount of RNA in the sample.

The invention also relates to the use of the kit according to the invention for the normalization of the amount of a target RNA sequence in a sample to the total amount of RNA in the sample.

The invention also relates to the use of the method according to the invention or the kit according to the invention for gene expression analysis.

In some embodiments, the means and methods according to the present invention are used for the normalization of gene expression levels.

Preferably, the quantities of two or more nucleic acids to be quantified are normalized simultaneously, i.e. at the same time.

In some embodiments, one or more of the components are premixed in the same reaction container.

### EXAMPLES

### Example 1

### Influence of Dye Concentration

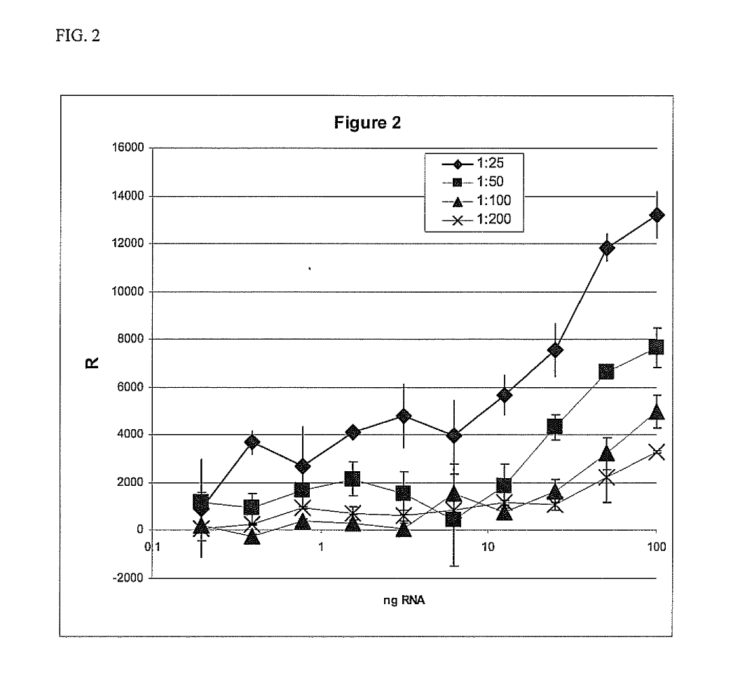

For RNA quantification using MTP based Fluorimeters Quant-IT RNA is usually diluted 1:200. To test the influence of the dye concentration when used for the detection of RNA by measuring the corresponding fluorescence in a PCR cycler, different Quant-IT RNA concentrations were tested. Total RNA was isolated from MCF7 cells using RNeasy Mini Spin columns. Different amounts of this RNA were used as template for SYBR Green based onestep RT-PCR using QIAGEN QuantiTect SYBR Green RT-PCR Kits. The RT-PCR Mastermix was supplemented with the Quant-IT RNA dye to yield final dilutions as indicated. Primers targeting ERK mRNA were used for amplification. One-step RT-PCR reaction was done in 96well PCR-Plates and a Stratagene MX3005P thermal cycler. Reactions were performed in duplicates. Fluorescence of control reactions without RNA were substracted as blanks, with separate blanks for each Quant-IT RNA dilution; see FIG. 2. With all tested Quant-IT RNA concentration a linear relationship between the log of RNA concentration and the cT values was observed. With the highest dye concentration (1:25) the cTs were increased by ˜1. However, the slope, an indicator of PCR amplification efficiency, is not changed by addition of Quant-IT RNA. This shows that SYBR Green based detection and quantification is possible in the presence of even high concentrations of Quant-IT RNA.

### Example 2

### Dilution of Dye

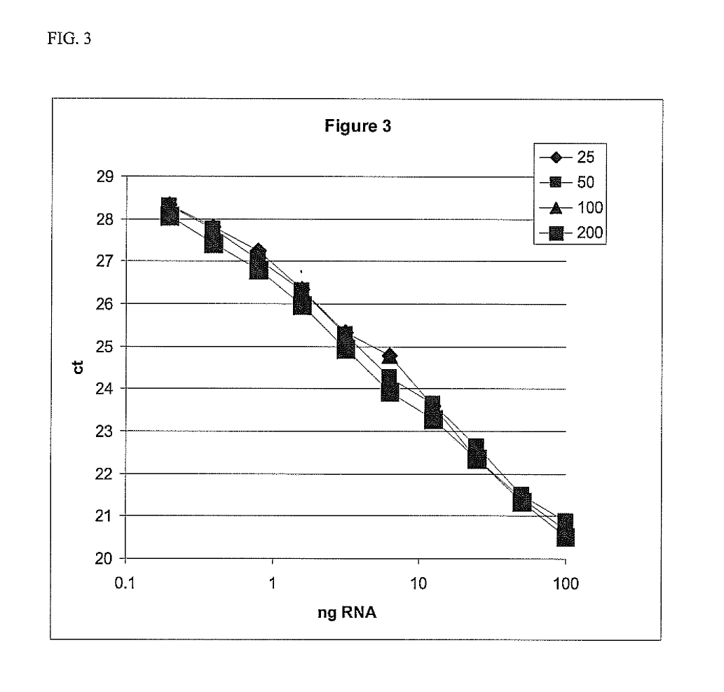

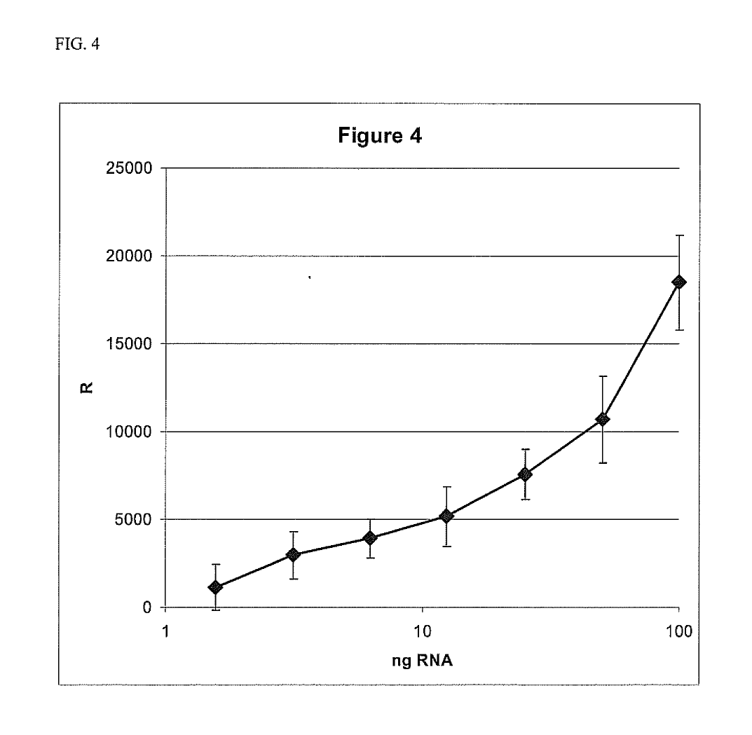

A similar experiment as in example 1 was done with the following changes: Quant-IT RNA was used only in a final dilution of 1:25. Each amount of RNA was tested in 12 fold replication to test the variability of the assay. Mean fluorescence of the blanks containing no RNA were substracted. The FIGS. 3 and 4 show the results for the Quant-IT RNA measurement and the SYBRGreen based detection.

### Example 3

### RNA Concentrations of 10 to 100 ng

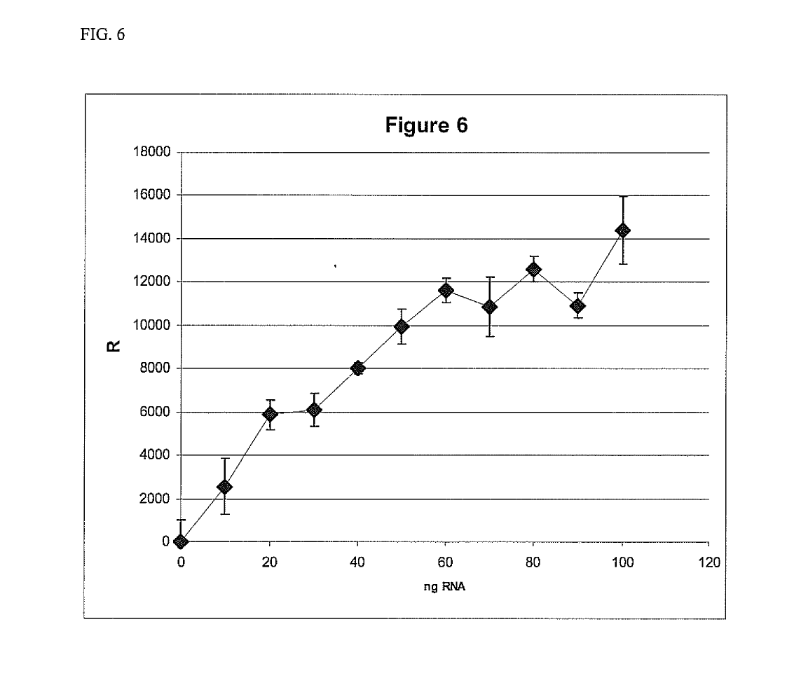

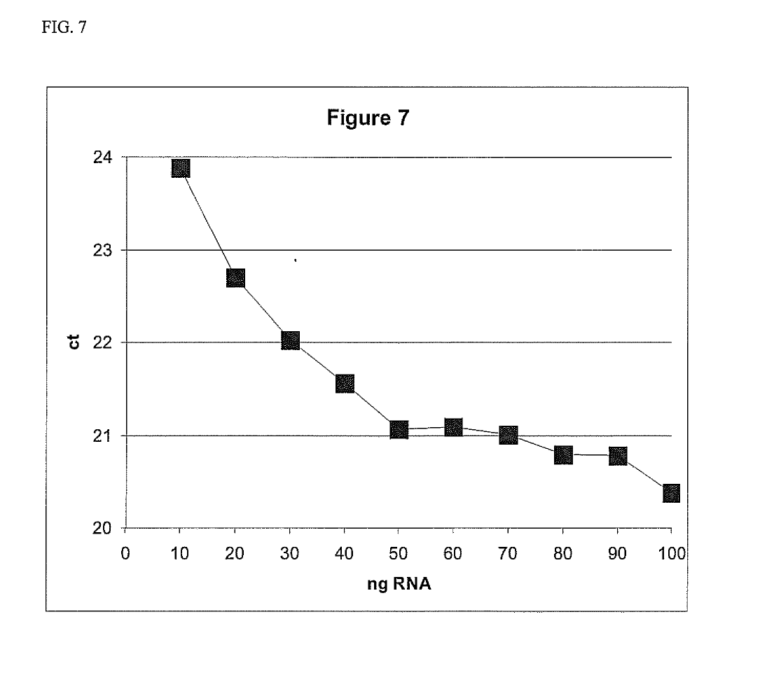

While in the previous examples RNA concentrations covering 2 orders of magnitude were used, the FIGS. 6 and 7 show results form an experiment where RNA concentrations of 10-100 ng per reaction with along spacing where used.

### DESCRIPTION OF DRAWINGS

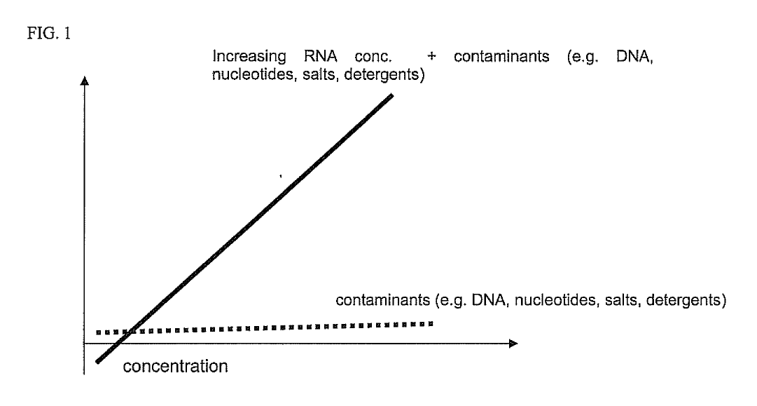

### FIG. 1

To allow the quantification of RNA present in one-step RT-PCR reactions using SYBR Green based detection any fluorescent dye can be used that fulfils the two following requirements, (i) spectral properties do not interfere with the spectral properties used for detection of the Gene of Interest (GoI). The GoI is predominantly detected using the fluorescent dye SYBR Green. SYBR Green binds to double-stranded DNA and the resulting to DNA-dye-complex has an excitation maximum at 488 nm and an emission maximum at 522 nm and (ii) the reaction mix used for one-step RT-PCR contains various substance that can interfere with the measurement of RNA: Nucleotides, Oligonucleotides, Proteins, Detergents, Salts. The fluorescent dye of choice must therefore not be influenced by such substances to grade that disturbs the quantification, but needs to be highly sensitive for RNA. An example of such a fluorescent dye is Quant-IT RNA, sold by Invitrogen. It has excitation/emission maxima of 644/673 nm when bound to RNA and does therefore not interfere with SYBR Green measurements. It is highly specific for RNA it is not disturbed by substance usually present in one-step RT-PCR reaction.

### FIG. 2

The figure shows increasing fluorescence with increasing amounts of RNA spiked into the RT-PCR reaction. Increasing the concentration of Quant-IT RNA increases the absolute fluorescence, but also the signal to noise ratio.

### FIG. 3

The cT values obtained from the SYBR Green fluorescence values in the same PCR run are plotted against the amount of RNA in FIG. 3.

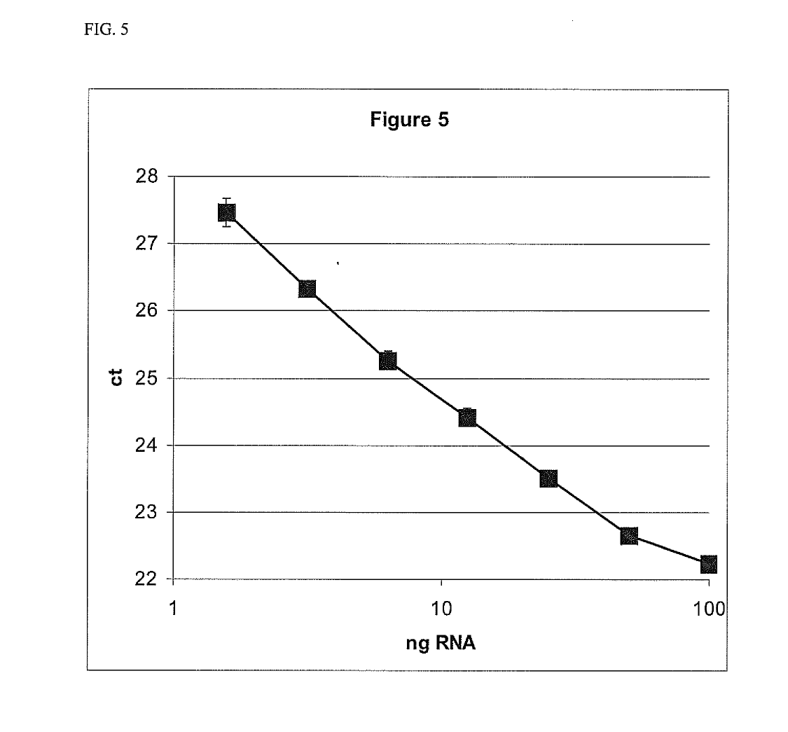

### FIGS. 4 AND 5

Again, increasing Quant-IT RNA fluorescence with increasing amounts of RNA was found. The SYBR Green cTs decreased with increasing amounts of RNA in the presence of the Quant-It RNA dye. This shows that it is possible to quantify the amount of RNA spiked into a PCR reaction using a specific fluorescence dye and to do SYBR Green based amplification and detection of a specific RNA target in the same reaction and using the same instrumentation (PCR Cycler).

### FIGS. 6 AND 7

Also in this narrower dilution series it was possible quantify the amount of RNA per PCR reaction. The SYBR Green cT values from the same multiplexed reaction showed corresponding decreasing cTs with increasing concentration of RNA. Because of the linear scale of the x-axis in this experiment the cT curve show a log-like characteristic.

1. Method for the quantification of one or more target ribonucleic acids in a sample comprising the steps of:
(i) providing a sample comprising said one or more target ribonucleic acids;
(ii) contacting said sample with a ribonucleic acid specific fluorescence dye under conditions allowing for binding of said dye to the one or more ribonucleic acids in said sample;
(iii) measuring fluorescence of said RNA-bound dye in said sample;
(iv) correlating said measured fluorescence to the total amount of RNA in the sample;
(v) reverse transcribing said one or more ribonucleic acids, thereby creating double-stranded nucleic acids;
(vi) amplifying said one or more created double-stranded nucleic acids, wherein one or more fluorescence probes specific for said one or more amplification products are present during and/or after amplification under conditions allowing for binding of said one or more probes to said one or more created double-stranded deoxyribonucleic acids in the sample;
(vii) measuring fluorescence of said one or more probes bound to said one or more amplification products during and/or after the amplification reaction and correlating said measured fluorescence to the amount of target RNA sequence in the sample;
(viii) normalizing the amount of target RNA sequence in the sample to the total amount of RNA in the sample.

2. Method according to claim 1, wherein the reaction takes place in one reaction vessel.

3. Method according to claim 1, wherein the sample is total RNA preparation.

4. Method according to claim 1, wherein the RNA specific fluorescence dye is Quant-iT™-RNA reagent.

5. Method according to claim 1, wherein the fluorescence probe specific for DNA is a fluorescence dye specific for double-stranded DNA.

6. Method according to claim 5, wherein the fluorescence dye is selected from the group comprising SYBR Green I, SYTO-9, SYTO-13, SYTO-16, SYTO-64, SYTO-82, YO-PRO-1, SYTO-60, SYTO-62, SYTOX Orange, SYBR Green I, TO-PRO-3, TOTO-3, POPO-3 and BOBO-3.

7. Method according to claim 1, wherein the fluorescence probe specific for DNA is an oligonucleotide probe labelled with a fluorescence dye, wherein the oligonucleotide probe is substantially complementary to a sequence on the DNA created from the target RNA sequence.

8. Method according to claim 1, wherein two or more target ribonucleic acids are quantified in the sample and wherein an oligonucleotide probe labelled with a different fluorescent dye is used for each target RNA to be quantified.

9. Method according to claim 1, wherein the amplification reaction is a polymerase chain reaction.

10. Method according to claim 9, wherein the polymerase chain reaction is a quantitative real-time PCR.

11. Kit for the quantification of target RNA in a sample comprising:
(i) a fluorescence dye which specifically binds to RNA,
(ii) one or more fluorescence probes specific for one or more DNA amplification products.

12. Use of an RNA specific fluorescence dye in the normalization of the amount of a target RNA sequence in a sample to the total amount of RNA in the sample.

13. Use of the kit according to claim 11 for the normalization of the amount of a target RNA sequence in a sample to the total amount of RNA in the sample.

14. Use of the method according to claim 1 for gene expression analysis.
